(19) United States
(12) Patent Application Publication
(10) Pub. No.: US 2022/0031282 A1
(43) Pub. Date: Feb. 3, 2022

(54) ULTRASONIC CT APPARATUS, ULTRASONIC IMAGE GENERATION METHOD, AND ULTRASONIC IMAGE GENERATION APPARATUS

(71) Applicant: Hitachi, Ltd., Tokyo (JP)

(72) Inventors: Atsuro SUZUKI, Tokyo (JP); Yushi TSUBOTA, Tokyo (JP); Takahide TERADA, Tokyo (JP); Kazuhiro YAMANAKA, Tokyo (JP); Kenichi KAWABATA, Tokyo (JP)

(21) Appl. No.: 17/333,192

(22) Filed: May 28, 2021

(30) Foreign Application Priority Data
Jul. 30, 2020 (JP) 2020-129431

(51) Int. Cl. A61B 8/13; A61B 8/00; A61B 8/08

(52) U.S. Cl. CPC A61B 8/13; A61B 8/0825; A61B 8/5207; A61B 8/4488

(57) ABSTRACT

A sound wave is transmitted to a subject from one or more transducers in an transducer array in which a plurality of the transducers are arranged. A measured sound pressure measured by a plurality of transducers for the sound wave transmitted through an imaging region of the subject is received from the transducers. The measured sound pressure is processed to generate a transmitted wave image in the imaging region. A simulated sound wave having a sound pressure changing with time is generated from a simulated sound source, the sound pressure when the simulated sound wave is transmitted through the imaging region and reaches a plurality of simulated detectors is obtained by calculation as a calculated sound pressure, and the transmitted wave image is sequentially corrected using the calculated sound pressure with the transmitted wave image as an initial image.

### CROSS-REFERENCE TO RELATED APPLICATION

The present application claims priority from Japanese application JP2020-129431, filed on Jul. 30, 2020, the contents of which is hereby incorporated by reference into this application.

### BACKGROUND OF THE INVENTION

### Field of the Invention

The present invention relates to an image reconstruction method for ultrasonic CT.

### Background Art

An ultrasonic CT (Computed Tomography) apparatus dedicated for breast is known as a medical diagnostic apparatus that applies ultrasonic measurement to detection of breast cancer (Patent Literature 1). In the ultrasonic CT apparatus, an transducer array, which is a transmitter and receiver of ultrasonic waves, is placed around the breast inserted into water, the ultrasonic wave transmitted through the breast is received in the entire circumferential direction, and an image showing distribution of sound speed and attenuation is reconstructed from a received signal. In general, the sound speed and attenuation of tissues are quantitative values, and the sound speed and the attenuation of a tumor are higher than those of normal tissues such as surrounding mammary glands and fats, so that the tumor can be quantitatively detected from a transmitted wave image.

As a method of capturing the transmitted wave image, a method of irradiating a subject with a diffused wave spreading at a predetermined angle from one sound source (transducer) to obtain a transmitted signal is known (Non-Patent Literature 1).

As an image reconstruction method for generating a transmitted wave image from the transmitted signal, a straight-ray method and a bent-ray method are known. The straight-ray method is a method for reconstructing the image by approximating a trajectory of the ultrasonic wave to a straight line, and a calculation is fast but spatial resolution is low. The bent-ray method is a method for reconstructing the image in consideration of refraction of the ultrasonic wave, and the spatial resolution is higher than that of the straight-ray method.

Patent Literature 1 discloses an FWI (full waveform inversion) method as a method for reconstructing a sound speed image having a higher spatial resolution than that of the straight-ray method or the bent-ley method, and the FWI method is also applied to clinical data.

In the FWI method, the sound speed image is obtained from a measured transmitted signal by a related-art reconstruction method, to be used as an initial image, distribution of sound pressure is calculated from a certain sound source and the initial image by simulation, and the sound speed image is updated so that an error between this created simulation data (the distribution of sound pressure) and measurement data is reduced. Patent Literature 1 discloses a calculation algorithm of the FWI method.

### CITATION LIST

### Patent Literature

Patent Literature 1: U.S. Patent Application Publication No. 2016/0030000
Non-Patent Literature 1: Pratt, R., “Seismic waveform inversion in the frequency domain, Part 1: Theory and verification in a physical scale model.” GEOPHYSICS, 1999, 64(3), 888-901.

### SUMMARY OF THE INVENTION

### Technical Problem

In the FWI method, the sound source used for simulating the distribution of sound pressure in an imaging region generates a wave having a sound pressure changing with time similar to an actual sound source (the transducer). In order to match a wave signal emitted by a simulated sound source with a wave signal emitted by the actual sound source, a signal source scaling coefficient γ is calculated as described in paragraph 0045 in Patent Literature 1. Specifically, Patent Literature 1 discloses that estimation of the signal source scaling coefficient γ is treated as a linear estimation problem, and a complex scaling coefficient γ is calculated by dividing a product of the received signal of a measured value and the wave signal of simulation result by a square of the wave signal of the simulation result.

However, the above calculation of the scaling coefficient γ does not consider whether a phase of the wave generated by the actual sound source and a phase of the wave generated by the sound source at the time of simulation match each other.

If the phase of the wave generated from the sound source at the time of simulation is different from the phase of the wave generated by the actual sound source, estimation of an arrival time of the wave of the received signal may be deviated by up to one cycle. Therefore, it is important to accurately match the phases of the two in order to improve image quality of FWI.

However, since it is practically difficult to control the phase of the wave at the time of emission from an actual transducer with high accuracy, it is necessary to accurately estimate the phase of the wave emitted by the sound source in the simulation so as to match the phase of the wave of the actual sound source.

An object of the present invention is to reduce deviation of waveform between a sound wave generated from a simulated sound source by simulation and a sound wave emitted by the transducer, and to obtain a high-resolution reconstructed image in the FWI method.

### Means for Solving the Problems

In order to achieve the above object, according to the present invention, an ultrasonic CT apparatus includes: a transmitter that transmits a sound wave to a subject from one or more transducers in an transducer array in which a plurality of transducers are arranged; a receiver that receives a measured sound pressure measured by a plurality of transducers for the sound wave transmitted through an imaging region of the subject, from the transducers; an image reconstructor that processes the measured sound pressure to generate a transmitted wave image in the imaging region; and a sequential updater that causes a simulated sound source to generate a simulated sound wave having a sound pressure changing with time, obtains by calculation the sound pressure when the simulated sound wave is transmitted through the imaging region and reaches a plurality of simulated detectors, as a calculated sound pressure, and sequentially corrects the transmitted wave image using the calculated sound pressure with the transmitted wave image as an initial image. The sequential updater uses the calculated sound pressure to perform arithmetic processing that brings a waveform of the simulated sound wave generated by the simulated sound source closer to a waveform of the sound wave transmitted by the transducers.

### Advantage of the Invention

According to the present invention, in the FWI method, change in the sound pressure of the wave generated from the simulated sound source by simulation can be brought close to the change in sound pressure of the sound wave emitted by the actual transducer, so that the high-resolution reconstructed image can be obtained.

### BRIEF DESCRIPTION OF THE DRAWINGS

FIG. 1 is a block diagram illustrating a configuration of an ultrasonic CT apparatus of Embodiment 1;

FIG. 2 is a perspective view of an transducer array 30 of the ultrasonic CT apparatus of Embodiment 1;

FIG. 3 is an explanatory diagram illustrating irradiation waves emitted from an transducer 3a;

FIG. 4A is an explanatory view illustrating a transmitted wave image (an initial image) reconstructed by the ultrasonic CT apparatus, FIG. 4B is an explanatory view illustrating that for the transmitted wave image, a simulated sound source 53 is set to irradiate a simulated sound 50, and a calculated sound pressure is obtained at a position of a simulated detector 54, FIG. 4C is a graph illustrating a difference between the calculated sound pressure and the measured sound pressure, FIG. 4D is an image of a correction coefficient Δc obtained from the difference between the calculated sound pressure and the measured sound pressure, FIG. 4E is a view illustrating a corrected image obtained by subtracting the correction coefficient Δc, and FIG. 4F is an explanatory view illustrating a final image;

FIG. 5 is a flowchart illustrating an overview of operation of the ultrasonic CT apparatus of Embodiment 1;

FIG. 6 is a flowchart illustrating a detailed operation of the ultrasonic CT apparatus of Embodiment 1;

FIG. 7 is a flowchart illustrating a calibration determination function of the ultrasonic CT apparatus of Embodiment 2;

FIG. 8 is an example of a display screen illustrating a determination result of the calibration determination function of the ultrasonic CT apparatus of Embodiment 2; and

FIG. 9 is an example of a display screen illustrating a result of element deterioration determination by the calibration determination function of the ultrasonic CT apparatus of Embodiment 2.

### DESCRIPTION OF THE PREFERRED EMBODIMENTS

Hereinafter, an embodiment of the present invention will be described with reference to the drawings.

### Embodiment 1

### Overview

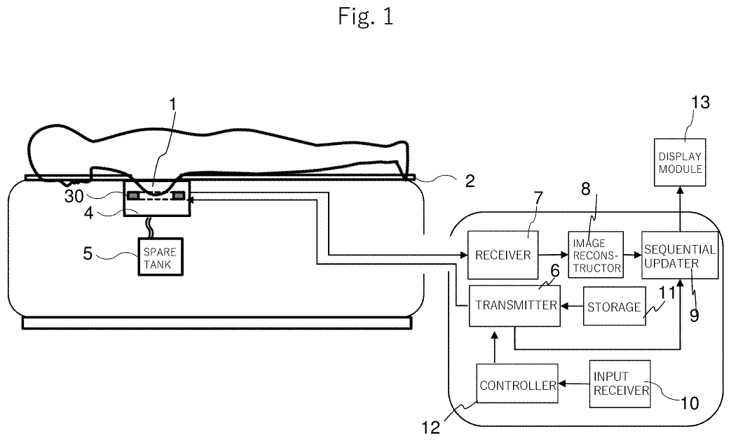

First, an overview of an ultrasonic CT apparatus of Embodiment 1 will be described with reference to FIG. 1 and the like.

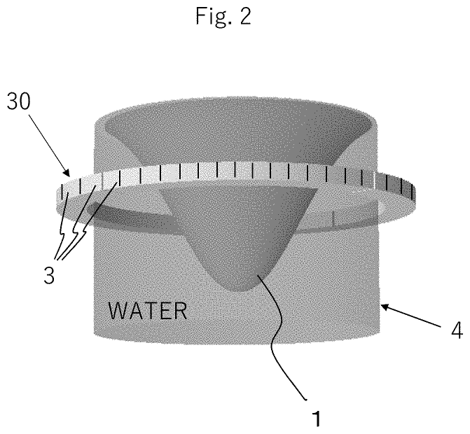

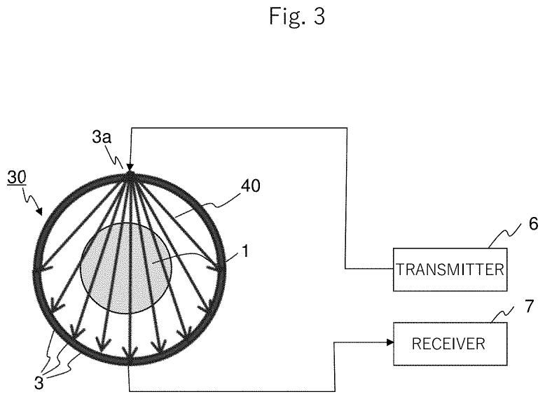

FIG. 1 is a diagram illustrating an overall structure of the ultrasonic CT apparatus, FIG. 2 is a perspective view of a breast of a subject 1 inserted in a water tank 4, and an transducer array 30, and FIG. 3 is a diagram illustrating irradiation of ultrasonic waves 40 from an transducer array 30. Here, in order to explain the ultrasonic CT apparatus suitable for breast cancer screening, an example of generating an image of the breast will be described, but an imaging target is not limited to the breast. Further, the present invention is not limited to the ultrasonic CT apparatus for obtaining a tomographic image of acoustic information of a living body, and is also applicable to, for example, obtaining the tomographic image of acoustic information of a stratum as shown in Non-Patent Literature 1. Therefore, in the present invention, the ultrasonic CT apparatus is defined as an apparatus that obtains the tomographic image of acoustic information of a substance.

As illustrated in FIG. 1, the ultrasonic CT apparatus of Embodiment 1 is configured to include a transmitter 6, a receiver 7, an image reconstructor 8, a sequential updater 9, an input receiver 10, a storage 11, a controller 12 that controls an overall operation. The transmitter 6 and the receiver 7 are connected to the transducer array 30 in which a plurality of transducers 3 are arranged in a predetermined shape (for example, a ring shape) as illustrated in FIG. 2. A display module 13 is connected to the sequential updater 9.

The transducer array 30 is placed inside or outside the columnar water tank 4 as illustrated in FIG. 2. The water tank 4 is placed below an opening provided in a bed 2 on which the subject 1 is put (see FIG. 1). The subject 1 lies face down on the bed 2 and inserts the breast into the opening of the bed 2. Although not illustrated, a drive mechanism capable of translating the transducer array in an axial direction of the water tank 4 may be provided. The transducer 3 is an ultrasonic transmitter/receiver, and uses, for example, a piezoelectric element. The water tank 4 is filled with hot water, and is connected to a spare tank 5. The spare tank 5 has a function of purifying, overheating, and degassing the hot water in the water tank 4.

As illustrated in FIG. 3, the transmitter 6 outputs a transmission signal (an electrical signal) to one or more transducers 3a of the transducer array 30, and transmits sound waves (here, the ultrasonic waves) 40 from one or more transducers 3a. The sound wave 40 transmitted through an imaging region of the subject 1 reaches the transducers of the transducer array 30, and sound pressures thereof are measured. The receiver 7 receives the measured sound pressures (electrical signals) measured by the transducers 3. In the case of the ring-shaped transducer array 30, the imaging region is a region of the subject 1 located in a ring. The receiver 7 receives the measured sound pressures and performs A/D conversion.

The image reconstructor 8 processes the measured sound pressure to reconstruct the transmitted wave image in the imaging region. As the transmitted wave image, an image showing a physical property value distribution (for example, sound speed distribution and/or attenuation distribution) in the imaging region is generated by a known method.

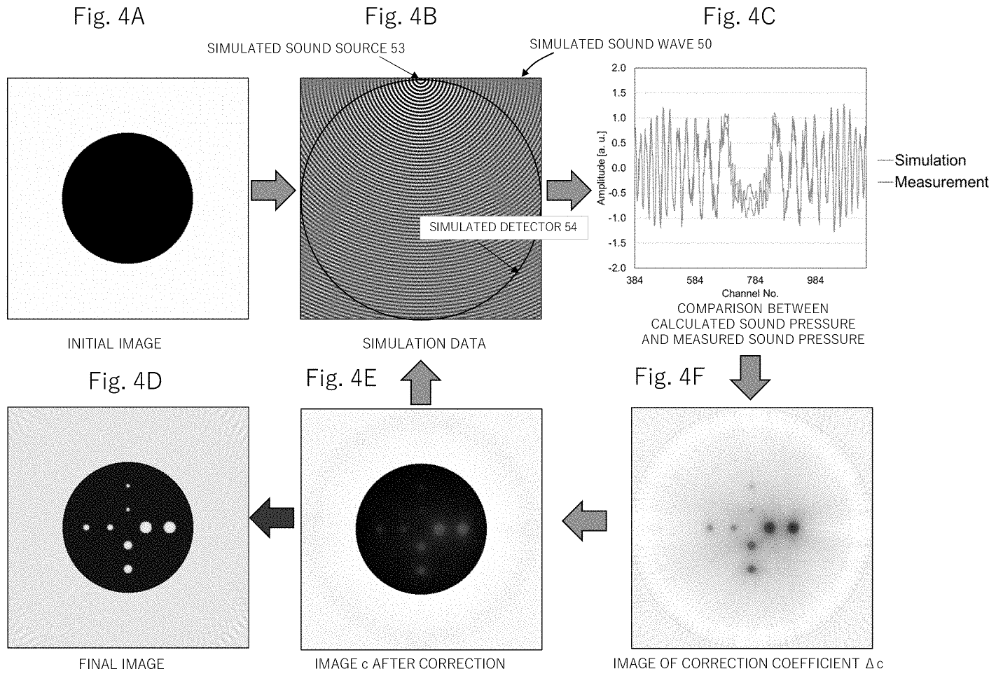

The sequential updater 9 uses the transmitted wave image generated by the image reconstructor 8 as an initial image (see FIG. 4A), and sequentially corrects the transmitted wave image using the sound pressure obtained by calculation. That is, the sequential updater 9 generates a simulated sound wave 50 (sound speed c) having sound pressure changing with time from a simulated sound source 53 (see FIG. 4B), and obtains by calculation the sound pressure (hereinafter referred to as a calculated sound pressure) when the simulated sound wave 50 is transmitted through the imaging region and reaches a plurality of simulated detectors 54. For example, as illustrated in FIG. 4C, the sequential updater 9 obtains a difference between the calculated sound pressure and the measured sound pressure, obtains a sound speed correction coefficient Δc (see FIG. 4D) that minimizes the difference, and sequentially corrects the transmitted wave image using the sound speed correction coefficient Δc (see FIGS. 4E and 4F).

At this time, in Embodiment 1, the sequential updater 9 uses the calculated sound pressure to perform a process of bringing a waveform of the simulated sound wave 50 generated by the simulated sound source 53 used for sequential correction closer to a waveform of the sound wave 40 transmitted by the transducer 3a.

Thus, in an FWI method, a sound pressure waveform of the sound wave 50 generated from the simulated sound source 53 by simulation can be brought close to a sound pressure waveform of the sound wave 40 emitted by an actual transducer 3a, so that a high-resolution reconstructed image can be obtained.

For example, the sequential updater 9 varies the waveform of temporal change in sound pressure of the simulated sound wave 50 into a plurality of types, obtains the calculated sound pressure for each of the plurality of types of waveforms, and selects the waveform of the simulated sound wave 50 generated by the simulated sound source 53 to be used for the sequential correction of the transmitted wave image based on a difference between the measured sound pressure and the calculated sound pressure.

The waveform of the simulated sound wave 50 may be selected only once before the above-mentioned sequential correction of the transmitted wave image, or may be selected each time the image is updated in a sequential correction process.

Note that the waveform of the simulated sound wave 50 referred to here includes any shape as long as it shows a time change of the sound pressure, for example, in addition to phase, maximum amplitude, and frequency, the time change of the wave may be a straight line (rectangular wave, triangle wave, or the like) or a curved line (function such as a sine wave), and if it is the curved line and has a different curve shape, the waveform is different.

For example, the sequential updater 9 is configured to prepare waveforms having different phases as the plurality of types of waveforms of the simulated sound wave 50, and select a waveform having a specific phase from the waveforms.

As a selection method, for example, the sequential updater 9 calculates a value of a predetermined function representing the difference between the measured sound pressure and the calculated sound pressure for the plurality of types of waveforms, and selects a waveform when the value of the function is minimized. As the function, for example, a function for calculating a square or a sum of squares of the difference between the measured sound pressure and the calculated sound pressure is used.

In the sequential updater 9, it is desirable that the simulated sound source 53 is placed at a position of the transducer 3a through which the transmitter 6 transmits the sound wave, and the simulated detector 54 is placed at a position of the transducer 3 where the receiver 7 receives the measured sound pressure.

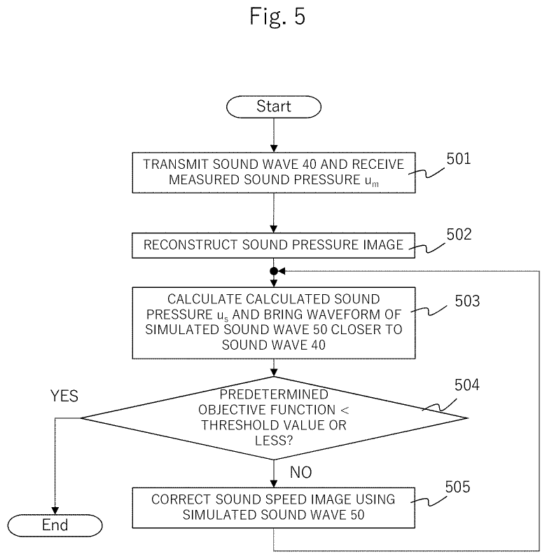

Hereinafter, the operation of each part of the ultrasonic CT apparatus will be described with reference to a flow shown in FIG. 5 and the like.

In the present embodiment, the image reconstructor 8 and the sequential updater 9 include a computer or the like provided with a processor such as a CPU (Central Processing Unit) or a GPU (Graphics Processing Unit) and a memory, and are configured such that the CPU reads and executes a program stored in the memory to realize functions of the image reconstructor 8 and the sequential updater 9 by software. However, the image reconstructor 8 and the sequential updater 9 can be partially or wholly implemented by hardware. For example, a custom IC such as an ASIC (Application Specific Integrated Circuit) or a programmable IC such as an FPGA (Field-Programmable Gate Array) may be used to configure the image reconstructor 8 and the sequential updater 9, and a circuit may be designed so as to implement the operation.

An overview of the operation of the ultrasonic CT apparatus will be described with reference to the flow of FIG. 5.

First, in Step 501, the transmitter 6 transmits the sound wave 40 from the transducer 3a to the subject 1. The sound wave 40 transmitted through the imaging region of the subject 1 is received by the transducer 3. The receiver 7 receives a measured sound pressure uₘ from the transducer 3.

In Step 502, the image reconstructor 8 uses the measured sound pressure uₘ to generate the transmitted wave image. Here, an example of generating a sound speed distribution image (hereinafter referred to as a sound speed image) as the transmitted wave image will be described below.

In Step 503, the sequential updater 9 calculates the calculated sound pressure uₛ detected by the simulated detector 54 when the simulated sound wave 50 is irradiated to the subject 1 from the simulated sound source 53, and uses the calculated sound pressure uₛ to perform the process of bringing the waveform of the simulated sound wave 50 closer to that of the sound wave 40.

In Step 504, when a value of a predetermined objective function is not smaller than a threshold value, the sequential updater 9 proceeds to Step 505 and corrects the sound speed image using the simulated sound wave 50 brought close to the sound wave 40 in Step 503.

The sequential updater 9 repeats Steps 503 to 505 until the value of the objective function is smaller than the threshold value in Step 504.

Thus, since the sound speed image can be corrected by using the simulated sound wave 50 having a waveform brought close to that of the sound wave 40, the high-resolution reconstructed image can be obtained with high accuracy.

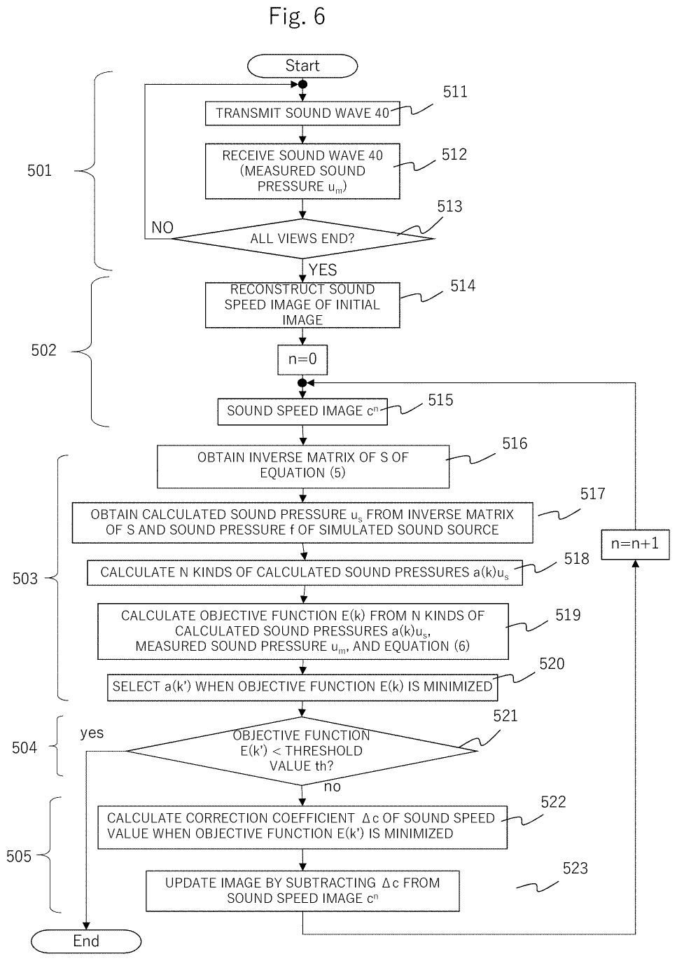

Details of processes of Steps 501 to 505 of FIG. 5 will be described with reference to the flow of FIG. 6.

### Step 501

A process of Step 501 will be described in more detail by Steps 511 to 513 of FIG. 6.

In Step 511, in the ultrasonic CT apparatus of the present embodiment, when the input receiver 10 receives an instruction to start imaging from a user, the transmitter 6 specifies for each view the transducer 3a stored in advance in the storage 11, and outputs the transmission signal having a predetermined frequency (about several MHz) to the transducer 3a. The transducer 3a that has received the transmission signal transmits the sound wave 40 to the subject 1.

In Step 512, a part of the sound wave 40 irradiated to the subject 1 is transmitted through the subject 1, received by the transducer 3, and converted into the electrical signal (measured sound pressure uₘ). The receiver 7 receives the measured sound pressure uₘ from the transducer 3 and performs processing such as A/D conversion.

Steps 511 and 512 transmit and receive the ultrasonic waves for all views (Step 513).

### Step 502

A process of Step 502 will be described in detail with reference to Steps 514 and 515 of FIG. 6.

In Step 514, the image reconstructor 8 generates the sound speed image in the subject 1 by performing an image reconstruction process using the measured sound pressure uₘ. In Step 515, the sound speed image is an initial image cⁿ (n=0) of a sequential update process performed by the sequential updater 9.

A specific example of the image reconstruction process will be briefly described. For example, the image reconstructor 8 can obtain the sound speed image by a straight-ray method. That is, the image reconstructor 8 performs Hilbert transform in a time direction for the measured sound pressure output by each transducer 3 in each view, and obtains a reception timing of the maximum amplitude of a received wave. The image reconstructor 8 similarly obtains the reception timing of the maximum amplitude for the measured sound pressure of each transducer 3 that has been received in advance before insertion of the subject 1. The image reconstructor 8 calculates a difference in reception timing before and after the insertion of the subject 1 for each view and each reception channel, and obtains a sinogram which is a set of these data. The image reconstructor 8 reconstructs the tomographic image by processing the sinogram of the difference in reception timing by a filtered back projection method (FBP) or the like. The tomographic image is a distribution image of the difference in “Slowness” of the ultrasonic waves before and after the insertion of the subject 1. The “Slowness” is a reciprocal of the sound speed. The image reconstructor 8 uses a sound speed value (an estimated value) of water to generate the sound speed distribution image (sound speed image) of the subject 1 from the distribution image of the difference in “Slowness”.

### Steps 503 to 505

In Step 503, the process of bringing the waveform of the simulated sound wave 50 to be used for the sequential update process of the sound speed image in Steps 504 and 505 closer to the waveform of the actually transmitted sound wave 40 is performed in Steps 516 to 520 of FIG. 6. In order to explain this, first, the principle of the sequential update process by the FWI method will be described.

### Principle of Sequential Update in FWI Method

The FWI method includes a sound pressure calculation algorithm that calculates the calculated sound pressure uₛ and an inverse problem algorithm that corrects the transmitted wave image so as to minimize the objective function.

In the sound pressure calculation algorithm, the simulated sound source 53 is placed at the position of the transducer 3a in a space represented by the sound speed image, the simulated sound wave 50 is generated from the simulated sound source 53, transmitted through the space represented by the sound speed image, and the sound pressure (calculated sound pressure uₛ) of the simulated sound wave 50 that has reached the simulated detector 54 placed at the position of the transducer 3 is obtained by simulation.

When the FWI is performed in a frequency domain, the simulated sound source 53 is represented by a complex number as in the following Equation (1).

[Equation 1]

f=A·exp{i·θ} (1)

Here, A is a complex number coefficient that adjusts intensity and phase of the simulated sound source, i is an imaginary number, and θ is the phase. An adjustment coefficient A of the simulated sound source can be obtained, for example, by a known Pratt signal estimation method (Non-Patent Literature 1).

For the sound pressure, for example, a method of solving the Helmholtz equation in the frequency domain by the finite difference method can be used. The Helmholtz equation in the frequency domain is represented by the following equation.

$$[ Equation 2 ]$$

$$( {∇}^{2} + \frac{{ω}^{2}}{{c ( r )}^{2}} ) u ( r , ω ) = f ( r ) \qquad ( 2 )$$

Here, ω is an angular frequency and r is a position. c(r) is a pixel value (sound speed) of a pixel at a position r of the sound speed image (imaging region). u(r,ω) is a vector and a matrix representing the sound pressure at the position r of the sound speed image. f(r) is the simulated sound source represented by the function of Equation (1), and r is a position of the sound source.

In Equation (2),

[Equation 3]

∇² (3)

### is a Laplacian.

The space is discretized and Equation (2) is expressed by matrix representation by the finite difference method as follows.

[Equation 4]

S(r)u(r)=f(r) or u(r)=S(r)⁻¹f(r) (4)

In Equation (4), S (r) is called an impedance matrix and represents a coefficient matrix of a sound pressure u(r) in the finite difference method. That is, as shown in Equation (5), S (r) represents the matrix in parentheses on the left side of the sound pressure u (r) in Equation (2). In Equation (4), the frequency ω is assumed to be constant, and ω is omitted.

$$[ Equation 5 ]$$

$$( {∇}^{2} + \frac{{ω}^{2}}{{c ( r )}^{2}} ) = S ( r ) \qquad ( 5 )$$

As is apparent from a second equation on the right side of Equation (4), the sound pressure u (r) at the position r of the sound speed image can be obtained by calculation from a sound source f(r) represented by Equation (1) and an inverse matrix S(r)⁻¹ of an impedance matrix S(r). The sound pressure at the position r obtained by calculation is represented by a matrix as the calculated sound pressure uₛ(r).

Next, as a solution of the inverse problem algorithm, an objective function E(c) which is a sum of residual squares of the calculated sound pressure uₛ (r) obtained by calculation of Equation (4) and the measured sound pressure uₘ measured in Step 512 is obtained from Equation (6), and is minimized.

$$[ Equation 6 ]$$

$$E ( c ) = \frac{1}{2} {( {u}_{s} ( c ) - {u}_{m} )}^{H} ( {u}_{s} ( c ) - {u}_{m} ) \qquad ( 6 )$$

In Equation (6), H represents Hermitian transpose.

In order to minimize the objective function E(c) of Equation (6), a pixel value c of the sound speed image can be sequentially corrected, for example, by a steepest descent method represented by the following Equation (7).

[Equation 7]

cⁿ⁺=cⁿ−α∇E(cⁿ) (7)

In Equation (7), n is the number of repetitions, cⁿ is the pixel value (sound speed or attenuation) on the sound speed image before correction (nth time), cⁿ⁺¹ is the pixel value on the transmitted wave image after correction (n+1th time), and a is a parameter for adjusting a correction amount called a step length.

Here, a second term on the right side of Equation (7) is represented by Δc as in Equation (8).

[Equation 8]

α∇E(cⁿ)=Δc (8)

In the above description, the sound speed image is corrected by the steepest descent method, but other algorithms such as a conjugate gradient method can also be used. Further, the FWI of the present embodiment can be performed in any domain of the frequency domain and a time domain.

### Step 503

Using the above principle, the process of bringing the waveform of the simulated sound wave 50 closer to the waveform of the sound wave 40 in Step 503 will be specifically described with reference to Steps 516 to 520 of FIG. 6.

First, in Step 516, the sequential updater 9 calculates the impedance matrix S (r) by Equation (5) from the sound speed c (r) (r is a position of the pixel) indicated by the pixel value of the sound speed image in Step 515, and calculates the inverse matrix S (r)⁻¹.

In Step 517, the simulated sound source 53 is represented by f by Equation (1). The sequential updater 9 calculates the calculated sound pressure uₛ at a position of the simulated detector 54 from the calculated S (r)⁻¹, f which is the simulated sound source 53, and the second equation on the right side of Equation (4).

Next, in Step 518, the sequential updater 9 generates N kinds of calculated sound pressures a(k)uₛ by multiplying the calculated sound pressure uₛ of the simulated sound source 53 by each of N kinds of coefficients a(k) (k=0, 1, . . . , N−1) represented by Equation (9). As is apparent from Equation (9), the N types of coefficients a(k) are complex numbers having different angles θk, and are coefficients that act to shift the phase of the calculated sound pressure uₛ.

$$[ Equation 9 ]$$

$$a ( k ) = cos ( {θ}_{k} ) + i \cdot sin ( {θ}_{k} ) , {θ}_{k} = \frac{2 π}{N} \cdot k , \\ k = 0 , 1 , ⋯ , N - 1 \qquad ( 9 )$$

Next, in Step 519, the sequential updater 9 calculates the objective functions E(k) from N kinds of calculated sound pressures a(k)uₛ calculated in Step 518 and the measured sound pressure uₘ measured in Step 512 by Equation (6′) in which the calculated sound pressure uₛ(c) in Equation (6) is replaced with the calculated sound pressure a(k)uₛ. Thus, N kinds of objective functions E(k) can be obtained.

$$[ Equation 10 ]$$

$$E ( k ) = \frac{1}{2} {( a ( k ) {u}_{s} - {u}_{m} )}^{H} ( a ( k ) {u}_{s} - {u}_{m} ) \qquad ( {6}^{′} )$$

In Step 520, the sequential updater 9 selects the objective function E(k′) which is the minimum value among N types of objective functions E(k) (k′ is a value of k when the objective function E(k) is the minimum value), and selects a coefficient a(k′) when the objective function E(k′) of the minimum value is obtained.

From the second equation on the right side of the above Equation (4), when the impedance matrix S(r) is the same, that is, when the subject is the same subject 1, Equation (4) holds even if both sides of the second equation of Equation (4) are multiplied by a(k′). Therefore, it is understood that a(k′)uₛ obtained by multiplying the calculated sound pressure uₛ by a(k′) is obtained by a sound source a(k′)f obtained by multiplying the simulated sound source 53 represented by f by a(k′). That is, the sound pressure a(k′)uₛ, which is the minimum objective function E(k′), is obtained by the sound source a(k′)f having a phase of the simulated sound source 53 adjusted.

In this way, by the above Steps 516 to 520, it is possible to obtain the sound source a(k′)f having the phase of the simulated sound source 53 adjusted, which can bring the sound pressure waveform. 50 generated from the simulated sound source 53 closest to a phase of the sound wave 40 generated by the transducer 3a.

### Step 504

A process of obtaining the objective function in Step 504 of FIG. 5 will be described in Step 521 of FIG. 6.

In Step 521, the sequential updater 9 calculates the objective function E(k′) from the calculated sound pressure a(k′)uₛ, the measured sound pressure uₘ obtained in Step 512, and Equation (6). If the calculated objective function E(k′) is smaller than a predetermined threshold value, the sequential updater 9 ends the process, and the sound speed image cⁿ in Step 515 becomes the final image. If the objective function E(k′) is greater than the predetermined threshold value, the sequential updater 9 proceeds to Step 505 (Steps 522 to 523 in FIG. 6) to correct the sound speed image.

### Step 505

The correction process of the sound speed image in Step 505 of FIG. 5 will be described in Steps 522 to 523 of FIG. 6.

In Step 522, the sequential updater 9 calculates the sound speed c that minimizes E(c), for example, by the steepest descent method. Specifically, the correction coefficient Δc is calculated by Equation (8) for each pixel of the sound speed image.

In Step 523, the sequential updater 9 calculates a corrected pixel value cⁿ⁺¹ by subtracting Δc from the pixel value cⁿ as in Equation (7). Thus, the corrected sound speed image cⁿ⁺¹ is obtained.

The sequential updater 9 returns to Step 515, replaces the sound speed image cⁿ with the corrected sound speed image cⁿ⁺¹ calculated in Step 523, and sequentially corrects the sound speed image by repeating Steps 515 to 523 until the objective function E(k′) is smaller than a threshold value th in Step 521.

If the objective function E(k′) is smaller than the threshold value th in Step 521, the sequential updater 9 determines that the sequential correction has ended and displays the transmitted wave image on the display module 13.

As described above, in the present embodiment, in the FWI method, since the waveform (for example, the phase) of the sound wave 50 generated by the simulated sound source 53 can be sequentially brought closer to the sound wave 40 generated by the transducer 3a, it is possible to generate the sound speed image having a high spatial resolution.

In the flow of FIGS. 5 and 6, a configuration has been described in which Step 503 (Steps 516 to 520) is performed to obtain the coefficient a(k′) for each sequential correction, but the coefficient a(k′) may be calculated only at the first time, and the sequential correction may be performed as in the related-art case at the second and subsequent times.

In Step 521, in the above-described embodiment, it is determined that the sequential correction has ended if the objective function E(c) is not more than the predetermined threshold value th, but other determination criteria can also be used. For example, when Steps 515 to 523 are repeated a predetermined number of times, it can be determined that the sequential correction has ended.

In the above-described embodiment, a configuration has been described in which the image reconstructor 8 generates the sound speed image as the transmitted wave image and sequentially corrects the sound speed image, but an attenuation distribution image (attenuation image) may be generated, and similarly the attenuation image may also be sequentially corrected.

By the receiver 7 collecting the measured sound pressure while the transducer array 30 is moved up and down (in the axial direction of the water tank 4), the image reconstructor 8 can obtain the transmitted wave image of the subject 1 as a three-dimensional image.

### Embodiment 2

The ultrasonic CT apparatus of Embodiment 2 will be described. The ultrasonic CT apparatus of Embodiment 2 has the same configuration as that of Embodiment 1, and has a calibration determination function in addition to the same functions as those of Embodiment 1.

In the simulation in the FWI, it is necessary to obtain in advance the position (coordinate) of each of the transducers 3 of the transducer array 30, and a delay time for each transducer 3 for calibrating a transmission/reception timing of the sound wave 40 caused by variation in function of elements constituting the transducer 3. These transducer positions and delay times are called calibration information and are obtained in advance by calibration measurement. When the calibration information (transducer position, delay time) is accurate, the measured sound pressure and the calculated sound pressure match accurately.

The ultrasonic CT apparatus of Embodiment 2 uses the calibration determination function to transmit and receive the sound wave 40 to and from water without placing the subject 1 to measure the measured sound pressure, and compare it with the calculated sound pressure. At this time, as in Embodiment 1, by performing the process of bringing the waveform of the simulated sound wave 50 generated from the simulated sound source 53 closer to the sound wave 40, it is possible to accurately determine whether the calibration information is accurate.

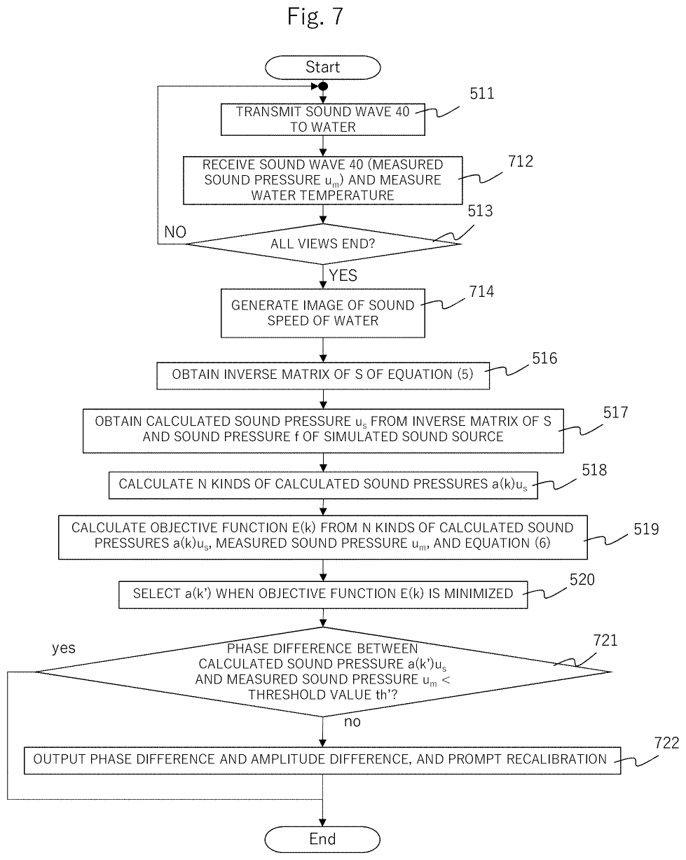

The operation of each part of the ultrasonic CT apparatus when determining whether the calibration information is accurate will be described with reference to the flow of FIG. 7. In the flow of FIG. 7, the steps of the same operation as the flow of FIG. 6 are denoted by the same numeral and will be briefly described.

In Step 511, the subject 1 is not placed in the water tank 4, and the sound wave 40 is transmitted from the transducer 3 only to the water, and in Step 712, the measured sound pressure uₘ is obtained and water temperature is recorded. Steps 511 and 712 are performed for all views.

In Step 714, the sound speed of the water is calculated from the water temperature measured in Step 712 by the equation of Del Grosso or the like, and the sound speed image having a calculated sound speed value uniformly is generated.

By performing Steps 517 to 520 as in Embodiment 1 using the generated sound speed image of the water, the coefficient a(k′) that brings the waveform of the sound wave 50 generated from the simulated sound source 53 closer to the sound wave 40 is selected.

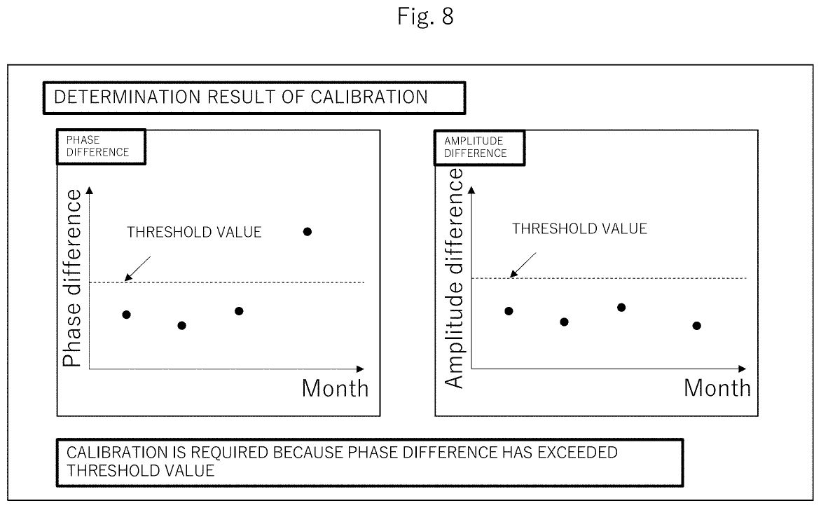

In Step 721, a phase difference between the calculated sound pressure a(k′)uₛ and the measured sound pressure uₘ when the water is measured in Step 712 is calculated. It can be determined that accuracy of the calibration information is higher as the phase difference is closer to 0, and the accuracy of the calibration information is lower as the phase difference is larger. Therefore, if the phase difference is not less than a predetermined threshold value th′, the process proceeds to Step 722, the phase difference between the calculated sound pressure a(k′)uₛ and the measured sound pressure uₘ, and an amplitude difference (a sound pressure difference) are displayed, for example, in the display module 13 as in FIG. 8, and a display prompting to perform the calibration is performed.

In this manner, in the apparatus of Embodiment 2, the accuracy of the calibration information can be determined as described above by measuring the water. By doing this on a regular basis (for example, once a month), it is possible to inform the user whether recalibration is necessary due to aging deterioration.

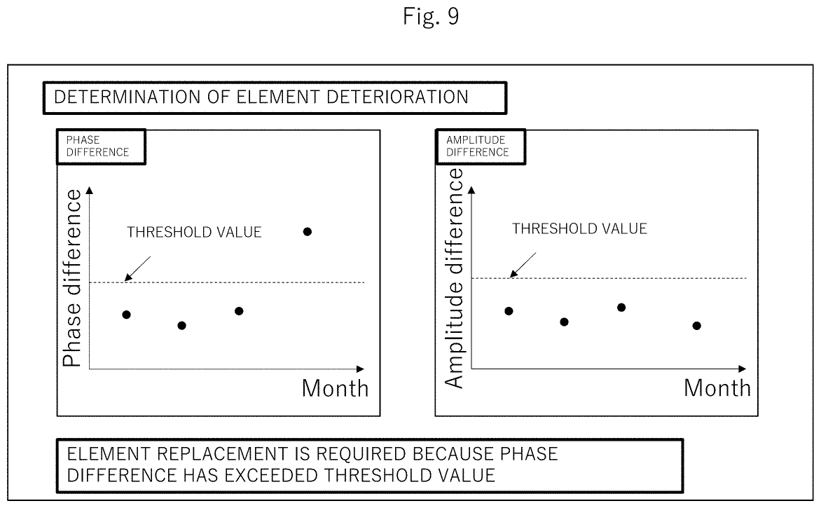

In Embodiment 2, since not only the phase difference between the calculated sound pressure a(k′)uₛ and the measured sound pressure uₘ, but also whether the amplitude difference (sound pressure difference) is not smaller than the threshold value can be determined, a display prompting element replacement can also be performed as in FIG. 9, by determining that the transducer 3 having the phase difference and/or the amplitude difference not smaller than the threshold value is a deteriorated defective element outputting an abnormal value.

Further, an output (the measured sound pressure uₘ) of the transducer 3 which is the defective element can be masked and not used for generating the image until the element is replaced. Specifically, in the flow of FIG. 6, in Steps 514, 519 and 521 in which the measured sound pressure uₘ is used, the measured sound pressure uₘ of the transducer 3 that is the defective element is masked and not used in the calculation.

In addition, the accuracy of the calibration information can be determined by performing the calibration measurement after installing the ultrasonic CT apparatus.

### DESCRIPTION OF REFERENCE NUMERALS AND SIGNS

1: subject, 2: bed, 3: transducer, 4: water tank, 5: spare tank, 6: transmitter, 7: receiver, 8: image reconstructor, 9: sequential updater, 10: input receiver, 11: storage, 12: controller, 13: display module.

What is claimed is:

1. An ultrasonic CT apparatus comprising:
a transmitter that transmits a sound wave to a subject from one or more transducers in an transducer array in which a plurality of transducers are arranged;
a receiver that receives a measured sound pressure measured by a plurality of transducers for the sound wave transmitted through an imaging region of the subject, from the transducers;
an image reconstructor that processes the measured sound pressure to generate a transmitted wave image in the imaging region; and
a sequential updater that causes a simulated sound source to generate a simulated sound wave having a sound pressure changing with time, obtains by calculation the sound pressure when the simulated sound wave is transmitted through the imaging region and reaches a plurality of simulated detectors, as a calculated sound pressure, and sequentially corrects the transmitted wave image using the calculated sound pressure with the transmitted wave image as an initial image, wherein
the sequential updater uses the calculated sound pressure to perform arithmetic processing that brings a waveform of the simulated sound wave generated by the simulated sound source closer to a waveform of the sound wave transmitted by the transducers.

2. The ultrasonic CT apparatus according to claim 1, wherein as the arithmetic processing, the sequential updater varies the waveform of temporal change in sound pressure of the simulated sound wave generated by the simulated sound source into a plurality of types, obtains the calculated sound pressure for each of the plurality of types of waveforms, and selects the waveform of the simulated sound wave used for sequential correction of the transmitted wave image from the plurality of types of waveforms based on a difference between the measured sound pressure and the calculated sound pressure.

3. The ultrasonic CT apparatus according to claim 2, wherein the plurality of types of waveforms of the simulated sound wave have different phases.

4. The ultrasonic CT apparatus according to claim 2, wherein the sequential updater calculates values of a predetermined function representing the difference between the measured sound pressure and the calculated sound pressure for the plurality of types of waveforms of the simulated sound wave, and selects a waveform when the value of the function is minimized.

5. The ultrasonic CT apparatus according to claim 4, wherein the sequential updater calculates a sum of squares of the difference between the measured sound pressure and the calculated sound pressure as the predetermined function.

6. The ultrasonic CT apparatus according to claim 2, wherein the sound pressures of the plurality of types of waveforms of the simulated sound wave are calculated from the sound source represented by a(k)f (where f is an arbitrary complex number, a(k) is a(k)=exp{i·θk}, θk=(2π/N)·k, k=0, 1, . . . , N−1, N is an arbitrary integer).

7. The ultrasonic CT apparatus according to claim 2, wherein when the sequential updater sequentially corrects the transmitted wave image, it obtains a sound speed correction coefficient that minimizes the difference between the measured sound pressure and the calculated sound pressure, and uses the sound speed correction coefficient to sequentially correct the transmitted wave image.

8. The ultrasonic CT apparatus according to claim 1, wherein the sequential updater places the simulated sound source at a position of the transducer from which the transmitter transmits the sound wave, and places the simulated detector at a position of the transducer from which the receiver receives the measured sound pressure.

9. The ultrasonic CT apparatus according to claim 1, wherein the sequential updater calculates at least one of a phase difference and a sound pressure difference between the measured sound pressure measured for water without placing the subject in the water and the calculated sound pressure obtained for the simulated sound wave after the arithmetic processing that brings the waveform of the simulated sound wave closer to the waveform of the sound wave transmitted by the transducers.

10. The ultrasonic CT apparatus according to claim 9, wherein when at least one of the phase difference and the sound pressure difference is not less than a predetermined threshold value, the sequential updater causes a display module to perform a display prompting to perform calibration.

11. The ultrasonic CT apparatus according to claim 9, wherein when at least one of the phase difference and the sound pressure difference is not less than a predetermined threshold value, the sequential updater masks the measured sound pressure having the phase difference and the sound pressure difference not less than the threshold value, and does not use the measured sound pressure to sequentially correct the transmitted wave image.

12. An ultrasonic image generation method comprising:
transmitting a sound wave to a subject from one or more transducers in an transducer array in which a plurality of transducers are arranged;
receiving a measured sound pressure measured by a plurality of transducers for the sound wave transmitted through an imaging region of the subject;
processing the measured sound pressure to generate a transmitted wave image in the imaging region;
causing a simulated sound source to generate a simulated sound wave having a sound pressure changing with time, obtaining by calculation the sound pressure when the simulated sound wave is transmitted through the imaging region and reaches a plurality of simulated detectors, as a calculated sound pressure;
using the calculated sound pressure to bring a waveform of the simulated sound wave generated by the simulated sound source closer to a waveform of the sound wave transmitted by the transducers; and
using the calculated sound pressure of the simulated sound wave that has been brought close to the waveform of the sound wave to sequentially correct the transmitted wave image with the transmitted wave image as an initial image.

13. An ultrasonic image generation apparatus comprising:
an image reconstructor that transmits a sound wave to a subject from one or more transducers in an transducer array in which a plurality of transducers are arranged, receives a measured sound pressure measured by a plurality of transducers for the sound wave transmitted through an imaging region of the subject, and processes the measured sound pressure to generate a transmitted wave image in the imaging region; and
a sequential updater that causes a simulated sound source to generate a simulated sound wave having a sound pressure changing with time, obtains by calculation the sound pressure when the simulated sound wave is transmitted through the imaging region and reaches a plurality of simulated detectors, as a calculated sound pressure, and sequentially corrects the transmitted wave image using the calculated sound pressure with the transmitted wave image as an initial image, wherein
the sequential updater uses the calculated sound pressure to perform arithmetic processing that brings a waveform of the simulated sound wave generated by the simulated sound source closer to a waveform of the sound wave transmitted by the transducers.
